(19) United States
(12) Patent Application Publication
(10) Pub. No.: US 2006/0183789 A1
(43) Pub. Date: Aug. 17, 2006

(54) Methods for preventing and treating obesity in patients with mc4 receptor mutations

(76) Inventors: Lavanya Rajachandran, Wallingford, CT (US); Elena Beretta, Groton, CT (US); James E Krause, Madison, CT (US)

Correspondence Address: NEUROGEN CORPORATION, 35 NORTHEAST INDUSTRIAL ROAD, BRANFORD, CT, 06405, US

(21) Appl. No.: 10/527,675

(22) PCT Filed: Sep. 23, 2003
(86) PCT No.: PCT/US03/29916

§ 371 Date: Dec. 7, 2005

Related U.S. Application Data
(60) Provisional application No. 60/413,321, filed on Sep. 25, 2002.

(51) Int. Cl. C12Q 1/68; A61K 31/405

(52) U.S. Cl. 514/419; 435/6

(57) ABSTRACT

Methods are provided for treating health conditions associated with altered MC4 receptor activity with melanin concentrating hormone receptor antagonists. Such compounds may be used, for example, to treat or prevent obesity and/or overeating, and to reduce body mass index, in patients carrying one or more MC4 receptor mutations.

### FIELD OF THE INVENTION

This invention relates generally to methods for treating health conditions associated with altered MC4 receptor activity, and more specifically to the use of melanin concentrating hormone receptor antagonists for the prevention and treatment of obesity and overeating in patients carrying MC4 receptor mutations.

### DESCRIPTION OF THE SEQUENCE LISTING

SEQ ID NO:1 Human MC4R amino acid sequence (Gantz et al. (1993) J. Biol. Chem. 268:15174-79)

### BACKGROUND OF THE INVENTION

Obesity is the most common nutritional problem in developed countries. By some estimates, obesity affects more than half of the population of the United States, where about 300,000 deaths annually are attributable to this condition. Obesity often leads to serious health conditions, such as diabetes, atherosclerosis, pulmonary embolism, coronary artery disease, hypertension, stroke, diabetes, sleep apnea, deep-vein thrombosis, hyperlipidemia and some cancers, and complicates numerous chronic conditions such as respiratory diseases, osteoarthritis, osteoporosis, gall bladder disease and dyslipidemias. Fortunately, however, many of the conditions caused or exacerbated by obesity can be resolved or dramatically improved by weight loss.

Once considered merely a behavioral problem (i.e., the result of voluntary hyperphagia), obesity is now recognized as a complex multifactorial disease involving defective regulation of food intake, food-induced energy expenditure and the balance between lipid and lean body anabolism. Both environmental and genetic factors play a role in the development of obesity. As a result, treatment programs that focus entirely on behavior modification have limited efficacy and are associated with recidivism rates exceeding 95%. Pharmacotherapy is now seen as a critical component of weight loss and subsequent weight management.

The central melanocortin system is critical for the regulation of food intake and energy balance. Within this system, melanocortins (a variety of different peptide products resulting from post-translational processing of pro-opiomelanocortin) stimulate or inhibit food intake via action at one or more melanocortin receptors. Alterations in melanocortin receptor activity have been shown to affect food intake.

Five melanocortin receptor subtypes have been described to date. Of these, melanocortin 4 receptor (MC4R) is the most abundant and most widely distributed in the brain. MC4R plays a specific role in appetite regulation. In both humans and mice, interruption of signaling at MC4R results in overeating, increased body-mass index and obesity. In addition, a variety of mutations in MC4R have been shown to cause morbid obesity in humans. Most known genetic mutations that result in obesity are recessive and cause only rare forms of obesity that occur in combination with endocrine abnormalities. Mutations in MC4R, however, can be dominant and are the most frequent known cause of severe obesity, estimated to occur in 3-5% of obese patients. MC4R is a 332-amino acid protein that belongs to the family of seven transmembrane G protein-coupled receptors (GPCR) and signals via adenylate cyclase. This receptor is expressed primarily throughout the brain, and is activated by a melanocortin peptide known as alpha-melanocyte stimulating hormone (alphaMSH). MC4R agonists such as alphaMSH have been shown to reduce food intake (i.e., they produce an anorexigenic effect), while antagonists of this receptor stimulate food intake (i.e., they produce an orexigenic effect).

Unfortunately, peptides such as alphaMSH are typically broken down by the digestive system, so that peptides are not usually suitable for oral administration to patients. In this regard, so-called small molecule pharmaceutical agents often have the advantage of being suitable for oral administration.

Other signaling pathways also contribute to obesity. Melanin concentrating hormone, or MCH, is a cyclic 19 amino acid hypothalamic peptide that functions as a regulator of food intake and energy balance, serving as a neurotransmitter in the lateral and posterior hypothalamus. MCH mRNA is overexpressed in ob/ob C57BL/6J mice, and mice with a targeted deletion of the MCH gene are characterized by reduced body weight, due to decreased feeding and increased metabolic rate. ICV administration (i.e., injection directly into the ventricles of the brain) of MCH has been shown to produce a mild orexigenic effect in rodents.

MCH activity is mediated via binding to specific receptors, of which MCH type 1 (MCHR1) and type 2 (MCHR2) receptors have been identified. MCHR1 is a 353 amino acid, 7-transmembrane, alpha-helical, G-coupled protein receptor, initially reported by Kolakowski et al. (1996) FEBS Lett. 398:253-58; Lakaye et al. (1998) Biochim. Biophys. Acta 1401:216-220; Chambers et al. (1999) Nature 400:261-65; and Saito et al. (1999) Nature 400:265-69. Upon binding MCH, MCHR1 receptors expressed in HEK 293 cell mediate a dose dependent release of intracellular calcium. Cells expressing MCH receptors have also been shown to exhibit a pertussis toxin sensitive dose-dependent inhibition of forskolin-elevated cyclic AMP, indicating that the receptor couples to a G_{i/o} G-protein alpha subunit. MCHR2 (An et al. (2001) Proc. Natl. Acad. Sci. USA 98:7576-7581; Sailer et al. (2001) Proc. Natl. Acad. Sci. USA 98:7564-7569; Hill et al. (2001) J. Biol. Chem. 276:20125-20129; Mori et al. (2001) Biochem. Biophys. Res. Commun. 283:1013-1018) has an overall amino acid identity of more than 30% with MCHR1, and is detected in most regions of the brain, with an expression pattern similar to that of MCHR1.

Although dysfunctions of various neurotransmitter and hormonal signaling pathways are known to contribute to obesity, the interrelationships among these pathways are poorly understood. For example, it has not been known which pathways are upstream and which downstream of the MC4 system. In this regard, modulating the activity of a pathway upstream of MC4R would not be expected to correct the phenotype resulting from a genetic defect in MC4R, while modulating the activity of a pathway downstream of MC4R might be expected to have some impact. Additionally, such impact would be of unpredictable and indeterminate magnitude, at least in the absence of any experimental data from which to extrapolate.

As a result, attempts to identify agents that decrease food intake in patients with obesity-promoting MC4R mutations have focused on the identification of agents that specifically affect the expression or activity of MC4R. To date, however, no such agents have been developed and marketed for medical use. The current limited understanding of the molecular and genetic factors contributing to the development of obesity has hampered the search for effective agents capable of inhibiting food intake in individuals with diminished MC4 receptor activity.

Accordingly, there is a need in the art for methods, especially methods employing small molecule non-peptide agents, that are capable of inhibiting food intake in obese individuals carrying MC4 receptor gene mutations that are associated with diminished MC4 receptor activity. The present invention fulfills this need, and provides further related advantages.

### SUMMARY OF THE INVENTION

The present invention provides compositions and methods useful for the treatment of overeating or obesity in patients carrying an MC4R mutation. Compositions generally comprise an effective amount of one or more MCH receptor antagonists, in combination with a physiologically acceptable carrier or excipient.

Within certain aspects, the present invention provides methods for treating obesity in a mammalian patient. Such methods comprise determining whether or not the obese patient carries a melanocortin 4 receptor (MC4R) mutation that is associated with obesity and, if the patient carries such a mutation, administering an amount of a non-toxic melanin concentrating hormone (MCH) receptor antagonist effective to reduce either or both of (1) food consumption and/or (2) body mass index in the patient upon sustained administration.

Within further aspects, methods are provided for preventing a recrudescence of obesity in a mammalian patient. Such methods comprise determining whether or not the previously obese patient carries a melanocortin 4 receptor (MC4R) mutation that is associated with obesity and, if the patient carries such a mutation, administering to the patient an amount of a non-toxic melanin concentrating hormone (MCH) receptor antagonist effective to reduce either or both of (1) food consumption and/or (2) body mass index in the patient upon sustained administration.

Methods are further provided for preventing obesity in a mammalian patient. Such methods comprise determining whether or not the patient carries a melanocortin 4 receptor (MC4R) mutation that is associated with obesity and, if the patient carries such a mutation, administering to the patient an amount of a non-toxic melanin concentrating hormone (MCH) receptor antagonist effective to reduce either or both of (1) food consumption and/or (2) body mass index in the patient upon sustained administration.

Within further aspects, the present invention provides methods for treating or preventing obesity (e.g., preventing a recrudescence of obesity) in a patient with an MC4R mutation, comprising administering such an effective amount of a non-toxic MCH receptor antagonist to a patient previously determined to carry such a mutation.

These and other aspects of the present invention will become apparent upon reference to the following detailed description.

### BRIEF DESCRIPTION OF THE DRAWING

FIG. 1 is a graph illustrating the effect of an MCH receptor antagonist on food consumption in rats with reduced MC4 receptor activity. The unshaded bar indicates the amount of food, in grams, consumed in a two hour period by rats treated with vehicle (saline) alone. The lightly shaded bar indicates the amount of food consumed in a two hour period by rats treated with 6 nmol HSO14 (an MC4 receptor antagonist), administered by direct injection to the lateral ventricle. The dark bar indicates the amount of food consumed in a two hour period by rats treated with 20 mg/kg MCH receptor antagonist orally 30 minutes before ICV administration of HSO14.

### DETAILED DESCRIPTION OF THE INVENTION

As noted above, the present invention provides methods for use in preventing or treating overeating and/or obesity in patients with one or more MC4R mutations. Compositions useful in the methods provided herein generally comprise a non-toxic MCH receptor antagonist. Such compositions may be administered to a patient with an MC4R mutation, for example, to reduce food intake, BMI and/or obesity.

Terminology

A “patient” is any individual being considered for treatment with an MCH receptor antagonist. Patients include humans, as well as other mammals such as companion animals and livestock, and are either obese or are at risk for a recrudescence of obesity.

A “melanocortin 4 receptor (MC4R) gene” is a naturally-occurring nucleotide sequence that encodes MC4R (i.e., a G-protein coupled receptor that is activated by alphaMSH and comprises an amino acid sequence that is at least 90% identical to SEQ ID NO:1) or would so encode MC4R (as determined by the precise chromosomal location, association with specific flanking sequences, comparison with allelic sequences, or like indications of gene locus identity) but for the presence of one or more nonsense, missense, frameshift, insertion or deletion mutations. The determination as to whether a nucleotide sequence is at least 90% identical to SEQ ID NO:1 is made using only the portions of SEQ ID NO:1 that align with the predicted protein product of the patient's MC4R gene. In other words, the protein product predicted for the gene if all frameshifted coding regions (if any) were in frame and all inserted or deleted regions (if any) were not figured in to the calculation. Such a determination is made using, for example, a ClustalW alignment. The term “MC4R gene” encompasses both the coding region and any introns or upstream or downstream regions that are tightly linked to the MC4R locus.

A “melanocoltin 4 receptor (MC4R) gene” is a naturally-occurring nucleotide sequence that encodes an MC4R (i.e., a G-protein coupled receptor that comprises an amino acid sequence that is at least 90% identical to SEQ ID NO:1). The encoded MC4R sequence may be truncated relative to SEQ ID NO:1; in such cases, the percent identity is determined using only the portion of SEQ ID NO:1 that aligns with the MC4R encoded by the patients MC4R gene using, for example, a ClustalW alignment. The term “MC4R gene” encompasses both the coding region and any introns or upstream or downstream regions that are tightly linked to the MC4R locus.

Patients are said to “carry at least one MC4R mutation” if the nucleotide sequence of one or both of the patient's MC4R genes contains at least one sequence feature that results in a decrease in receptor function or is otherwise determined to be associated with obesity. An MC4R mutation may be located in an upstream region, coding region, intron or downstream region of an MC4R gene. An MC4R mutation is generally a sequence alteration (e.g., any nucleotide deletion, insertion, or substitution) or other modification (e.g., an altered methylation state) relative to a reference MC4R sequence for a non-obese member of the patient's species. An appropriate reference sequence for humans is the MC4R sequence available at GenBank Accession Number L08603, a translation of which is provided herein as SEQ ID NO:1, and appropriate reference sequences for other animals may be obtained using conventional molecular biological techniques, using the human sequence as a probe. A determination as to whether a patient carries at least one MC4R mutation may be performed using standard techniques, such as PCR or RFLP mapping, with or without isolation of the MC4R gene. If prior genetic testing has been done, such a determination may be conveniently made by review of the patient's medical chart.

A mutation is considered to be “associated with obesity” if the mutation is identified in one or more obese patients, and is present at a significantly lower frequency in a non-obese population (as determined by any standard parametric test of statistical significance). MC4R mutations currently known to be associated with obesity include, but are not limited to, frameshift mutations (e.g., deletion of CTCT at codon 211, resulting in a truncated protein, or insertion of four nucleotides at codon 244), nonsense mutations (e.g., at codon 35, resulting in a truncated protein), and missense mutations (e.g., resulting in amino acid substitution(s) at position 11, 18, 30, 37, 50, 58, 78, 98, 102, 103, 112, 137, 150, 165, 170, 250, 252, 274, 301 and/or 317). The present invention encompasses treatment of patients with any MC4R mutation(s) that are currently known or are subsequently determined to be associated with obesity.

As used herein, a patient is considered “obese” if the patient's body mass index is greater than 28. Body mass index (BMI) may be readily calculated using the following formula:
BMI=(weight in kg)/(Height in meters)²

The term “MCH receptor” refers to a naturally-occurring mammalian (e.g., human, dog, cat, or monkey) MCH type 1 or type 2 receptor such as the MCH type 1 receptor (MCHR1; e.g., Lakaye et al., supra) and the MCH type 2 receptor (MCHR2; An et al., supra; Sailer et al., supra; Hill et al., supra; Mori et al., supra). SEQ ID NOs:1 and 2 of WO 03/060475 recite the DNA and amino acid sequences, respectively, of a Cynomolgus macaque MCH1R.

A “MCH receptor antagonist” is a compound that detectably inhibits MCH binding to one or more MCH receptors and/or inhibits MCH receptor-mediated signal transduction, as measured using the representative assays provided in Examples 1 and 2 herein. Antagonists for use within the context of the present invention are generally non-toxic. Within certain embodiments, an MCH receptor antagonist has a relatively low molecular weight (e.g., less than 700 amu) and is multi-aryl (i.e., has a plurality of unfused or fused aryl groups), non-peptide and amino acid free. Such compounds include, but are not limited to, substituted analogues of benzimidazole, 1-benzyl-4-aryl-piperazine, 1-benzyl-4-aryl-piperidine, and phenylcycloalkylmethylamino and phenylalkenylamino compounds. An antagonist binds “specifically” to MCH receptor if it binds to an MCH receptor (total binding minus nonspecific binding) with a Kᵢ that is 10-fold, preferably 100-fold, and more preferably 1000-fold, less than the Kᵢ measured for MCH receptor antagonist binding to other G protein-coupled receptors. An antagonist binds with “high affinity” if the Kᵢ at an MCH receptor is less than 1 micromolar, preferably less than 500 nanomolar, 100 nanomolar or 10 nanomolar. MCH receptor antagonists preferably have minimal agonist activity (i.e., induce an increase in the basal activity of the MCH receptor that is less than 5% of the increase that would be induced by one EC₅₀ of MCH), and more preferably have no detectable agonist activity within the assay described in Example 3).

The term “nontoxic” as used herein shall be understood in a relative sense and is intended to refer to any substance that has been approved by the United States Food and Drug Administration (“FDA”) for administration to mammals (preferably humans) or, in keeping with established criteria, is susceptible to approval by the FDA for administration to mammals (preferably humans). In addition, a highly preferred nontoxic compound generally satisfies one or more of the following criteria: (1) does not substantially inhibit cellular ATP production; (2) does not significantly prolong heart QT intervals; (3) does not cause substantial liver enlargement, and (4) does not cause substantial release of liver enzymes.

As used herein, a compound that “does not substantially inhibit cellular ATP production” is a compound that satisfies the criteria set forth in Example 4, herein. In other words, cells treated as described in Example 4 with 100 μM of such a compound exhibit ATP levels that are at least 50% of the ATP levels detected in untreated cells. In more highly preferred embodiments, such cells exhibit ATP levels that are at least 80% of the ATP levels detected in untreated cells.

A compound that “does not significantly prolong heart QT intervals” is a compound that does not result in a statistically significant prolongation of heart QT intervals (as determined by electrocardiography) in guinea pigs, minipigs or dogs upon administration of twice the minimum dose yielding a therapeutically effective in vivo concentration. In certain preferred embodiments, a dose of 0.01, 0.05. 0.1, 0.5, 1, 5, 10, 40 or 50 mg/kg administered parenterally or orally does not result in a statistically significant prolongation of heart QT intervals. By “statistically significant” is meant results varying from control at the p<0.1 level or more preferably at the p<0.05 level of significance as measured using a standard parametric assay of statistical significance such as a student's T test.

A compound “does not cause substantial liver enlargement” if daily treatment of laboratory rodents (e.g., mice or rats) for 5-10 days with twice the minimum dose that yields a therapeutically effective in vivo concentration results in an increase in liver to body weight ratio that is no more than 100% over matched controls. In more highly preferred embodiments, such doses do not cause liver enlargement of more than 75% or 50% over matched controls. If non-rodent mammals (e.g., dogs) are used, such doses should not result in an increase of liver to body weight ratio of more than 50%, preferably not more than 25%, and more preferably not more than 10% over matched untreated controls. Preferred doses within such assays include 0.01, 0.05. 0.1, 0.5, 1, 5, 10, 40 or 50 mg/kg administered parenterally or orally.

Similarly, a compound “does not promote substantial release of liver enzymes” if administration of twice the minimum dose yielding a therapeutically effective in vivo concentration does not elevate serum levels of ALT, LDH or AST in laboratory rodents by more than 100% over matched mock-treated controls. In more highly preferred embodiments, such doses do not elevate such serum levels by more than 75% or 50% over matched controls. Alternately, a compound “does not promote substantial release of liver enzymes” if, in an in vitro hepatocyte assay, concentrations (in culture media or other such solutions that are contacted and incubated with hepatocytes in vitro) equivalent to two-fold the minimum in vivo therapeutic concentration of the compound do not cause detectable release of any of such liver enzymes into culture medium above baseline levels seen in media from matched mock-treated control cells. In more highly preferred embodiments, there is no detectable release of any of such liver enzymes into culture medium above baseline levels when such compound concentrations are five-fold, and preferably ten-fold the minimum in vivo therapeutic concentration of the compound.

A “prodrug” is a compound that may not be an MCH receptor antagonist, but is modified in vivo, following administration to a patient, to produce such an antagonist. For example, a prodrug may be an acylated derivative of an MCH receptor antagonist. Prodrugs include compounds wherein hydroxy, amine or sulfhydryl groups are bonded to any group that, when administered to a mammalian subject, cleaves to form a free hydroxyl, amino or sulfhydryl group, respectively. Examples of prodrugs include, but are not limited to, acetate, formate and benzoate derivatives of alcohol and amine functional groups within an MCH receptor antagonist.

Melanin Concentrating Hormone Receptor Antagonists

As noted above, the present invention provides compositions and methods useful for the treatment of overeating and obesity, and for reducing body mass index, in patients carrying an MC4R mutation. Compositions provided herein generally comprise a non-toxic melanin concentrating hormone (MCH) receptor antagonist. Such antagonists may be specific for a particular MCH receptor (e.g., type 1 or type 2), or may function at multiple MCH receptors. MCH receptor antagonists for use within the compositions provided herein are, within certain embodiments, low molecular weight (e.g., less than 700 amu), multi-aryl, non-peptide and amino acid free.

As noted above, MCH receptor antagonists for use herein detectably inhibit MCH binding to MCHR1 and/or MCHR2 receptor (as determined using a standard in vitro MCH receptor ligand binding assay and/or calcium mobilization assay) at submicromolar concentrations, preferably at nanomolar concentrations, and more preferably at subnanomolar concentrations. References herein to a “MCH receptor ligand binding assay” refer to the standard in vitro receptor binding assay provided in Example 2. Briefly, a competition assay may be performed in which an MCH receptor preparation is incubated with labeled (e.g., ¹²⁵I) MCH and unlabeled test compound. Within the assays provided herein, the MCH receptor used is preferably a mammalian MCHR1 or MCHR2 receptor, more preferably a human or monkey MCHR1 or MCHR2 receptor. The MCH receptor preparation may be, for example, a membrane preparation from HEK293 cells that recombinantly express a human MCH receptor (e.g., Genbank Accession No. Z86090), monkey MCHR1 receptor (such as the MCHR1 sequence provided in SEQ ID NO:1 of WO 03/060475), or human MCHR1/human beta-2-adrenergic chimeric receptor.

Incubation with an MCH receptor antagonist results in a decrease in the amount of label bound to the MCH receptor preparation, relative to the amount of label bound in the absence of the antagonist. Preferably, an MCH receptor antagonist exhibits a Kᵢ at an MCH receptor of less than 1 micromolar, binding specifically and with high affinity to an MCH receptor. More preferably, such a compound exhibits a Kᵢ at an MCH receptor of less than 500 nM, 100 nM, 20 nM or 10 nM, within an MCH receptor ligand binding assay as described in Example 2.

A representative calcium mobilization assay is provided in Example 3. Generally preferred MCH receptor antagonists exhibit EC₅₀ values of about 4 micromolar or less, more preferably 1 micromolar or less, still more preferably about 100 nanomolar or less, 10 nanomolar or less or 1 nanomolar or less within a standard in vitro MCH receptor mediated calcium mobilization assay, as provided in Example 3.

In certain embodiments, MCH receptor antagonists include substituted 1-benzyl-4-aryl piperazine and piperidine analogues, as described within pending U.S. patent application Ser. No. 10/152,189, filed May 21, 2002. The corresponding PCT application published as WO 02/094799 on Nov. 28, 2002. This disclosure is hereby incorporated herein by reference for its teaching of MCH receptor antagonists (pages 3-5, 20-25 and especially Table 1 at pages 74-107) and the preparation thereof (pages 29-42 and 50-73).

Within other embodiments, MCH receptor antagonists for use within the present compositions are substituted benzimidazole analogues as described within pending U.S. patent application Ser. No. 10/399,499, filed Jan. 9, 2003. The corresponding PCT application published as WO 03/060475 on Jul. 24, 2003. This disclosure is hereby incorporated herein by reference for its teaching of MCH receptor antagonists (pages 2-5, Table I (pages 14-19) and Table II (pages 38-48)) and the preparation thereof (pages 23-24 and 32-38).

Within further embodiments, compounds for use within the present invention are as described within pending U.S. patent application Ser. No. 10/399,111, filed Jan. 9, 2003. The corresponding PCT application published as WO 03/059289 on Jul. 24, 2003. This disclosure is hereby incorporated herein by reference for its teaching of MCH receptor antagonists (pages 3-4 and 31-50) and the preparation thereof (pages 19-20 and 28-31).

Within further embodiments, compounds for use within the present invention are as described within U.S. Pat. No. 6,569,861, which is hereby incorporated by reference for its teaching of phenylcycloalkylmethylamino and phenylalkenylamino MCH receptor antagonists (columns 3-9 and 18-19) and the preparation thereof (columns 16-18).

Still further MCH receptor antagonists are described, for example, within the following published applications: WO 03/035055; US2003/0077701; WO 03/033480; WO 03/033476; WO 03/015769; WO 03/028641; WO 03/013574; WO 03/004027; WO 02/094799; WO 02/089729; WO 02/083134; WO 02/076947; WO 02/076929; WO 02/057233; WO 02/051809 and WO 02/10146. It will be apparent that the above are illustrative examples of MCH receptor antagonists, and are not intended to limit the scope of the present invention.

As noted above, compositions of the present invention may encompass a pharmaceutically acceptable salt of an MCH receptor antagonist. As used herein, a “pharmaceutically acceptable salt” is an acid or base salt that is generally considered in the art to be suitable for use in contact with the tissues of human beings or animals without excessive toxicity, irritation, allergic response, or other problem or complication. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, sulfanilic, formic, toluenesulfonic, methanesulfonic, ethane disulfonic, 2-hydroxyethylsulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC—(CH₂)ₙ—COOH where n is 0-4, and the like. Similarly, pharmaceutically acceptable cations include, but are not limited to sodium, potassium, calcium, aluminum, lithium and ammonium. Those of ordinary skill in the art will recognize further pharmaceutically acceptable salts, including those listed by Remington's Pharmaceutical Sciences, 17th ed., Mack Publishing Company, Easton, Pa., p. 1418 (1985). Accordingly, the present disclosure should be construed to include all pharmaceutically acceptable salts of MCH receptor antagonists.

A wide variety of synthetic procedures are available for the preparation of pharmaceutically acceptable salts. In general, a pharmaceutically acceptable salt can be synthesized from a parent compound that contains a basic or acidic moiety by any conventional chemical method. Briefly, such salts can be prepared by reacting the free acid or base form of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred.

Prodrugs of MCH receptor antagonists may be prepared by modifying functional groups present in the compounds in such a way that the modifications are cleaved to the parent compounds. Prodrugs include compounds wherein hydroxy, amine or sulfhydryl groups are bonded to any group that, when administered to a mammalian subject, cleaves to form a free hydroxyl, amino, or sulfhydryl group, respectively. Examples of prodrugs include, but are not limited to, acetate, formate and benzoate derivatives of alcohol and amine functional groups within an MCH receptor antagonist. Preferred prodrugs include acylated derivatives. Those of ordinary skill in the art will recognize various synthetic methods that may be employed to prepare prodrugs of an MCH receptor antagonist.

Pharmaceutical Compositions

The practice of the present invention employs pharmaceutical compositions comprising an MCH receptor antagonist, together with at least one physiologically acceptable carrier or excipient. Pharmaceutical compositions may comprise, for example, water, buffers (e.g., neutral buffered saline or phosphate buffered saline), ethanol, mineral oil, vegetable oil, dimethylsulfoxide, carbohydrates (e.g., glucose, mannose, sucrose or dextrans), mannitol, proteins, adjuvants, polypeptides or amino acids such as glycine, antioxidants, chelating agents such as EDTA or glutathione and/or preservatives. Certain pharmaceutical compositions are formulated for oral delivery to humans or other animals (e.g., companion animals such as dogs).

If desired, other active ingredients may also be included, such as leptin, a leptin receptor agonist, a melanocortin receptor 4 (MC4) agonist, sibutramine, dexenfluramine, a growth hormone secretagogue, a beta-3 agonist, a 5HT-2 agonist, an orexin antagonist, a neuropeptide Y₁ or Y₅ antagonist, a galanin antagonist, a CCK agonist, a GLP-1 agonist and/or a corticotropin-releasing hormone agonist.

Pharmaceutical compositions may be formulated for any appropriate manner of administration, including, for example, topical, oral, nasal, rectal or parenteral administration. The term parenteral as used herein includes subcutaneous, intradermal, intravascular (e.g., intravenous), intramuscular, spinal, intracranial, intrathecal and intraperitoneal injection, as well as any similar injection or infusion technique. In certain embodiments, compositions in a form suitable for oral use are preferred. Such forms include, for example, tablets, troches, lozenges, aqueous or oily suspensions, dispersible powders or granules, emulsion, hard or soft capsules, or syrups or elixirs. Within yet other embodiments, compositions of the present invention may be formulated as a lyophilizate.

Compositions intended for oral use may further comprise one or more components such as sweetening agents, flavoring agents, coloring agents and preserving agents in order to provide appealing and palatable preparations. Tablets contain the active ingredient in admixture with physiologically acceptable excipients that are suitable for the manufacture of tablets. Such excipients include, for example, inert diluents (e.g., calcium carbonate, sodium carbonate, lactose, calcium phosphate or sodium phosphate), granulating and disintegrating agents (e.g., corn starch or alginic acid), binding agents (e.g., starch, gelatin or acacia) and lubricating agents (e.g., magnesium stearate, stearic acid or talc). The tablets may be uncoated or they may be coated by known techniques to delay disintegration and absorption in the gastrointestinal tract and thereby provide a sustained action over a longer period. For example, a time delay material such as glyceryl monosterate or glyceryl distearate may be employed.

Formulations for oral use may also be presented as hard gelatin capsules wherein the active ingredient is mixed with an inert solid diluent (e.g., calcium carbonate, calcium phosphate or kaolin), or as soft gelatin capsules wherein the active ingredient is mixed with water or an oil medium (e.g., peanut oil, liquid paraffin or olive oil).

Aqueous suspensions comprise the active materials in admixture with excipients suitable for the manufacture of aqueous suspensions. Such excipients are suspending agents (e.g., sodium carboxymethylcellulose, methylcellulose, hydropropylmethylcellulose, sodium alginate, polyvinylpyrrolidone, gum tragacanth and gum acacia); and dispersing or wetting agents (e.g., naturally-occurring phosphatides such as lecithin, condensation products of an alkylene oxide with fatty acids such as polyoxyethylene stearate, condensation products of ethylene oxide with long chain aliphatic alcohols such as heptadecaethyleneoxycetanol, condensation products of ethylene oxide with partial esters derived from fatty acids and a hexitol such as polyoxyethylene sorbitol monooleate, or condensation products of ethylene oxide with partial esters derived from fatty acids and hexitol anhydrides such as polyethylene sorbitan monooleate). Aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the active ingredients in a vegetable oil (e.g., arachis oil, olive oil, sesame oil or coconut oil) or in a mineral oil such as liquid paraffin. The oily suspensions may contain a thickening agent such as beeswax, hard paraffin or cetyl alcohol. Sweetening agents such as those set forth above, and/or flavoring agents may be added to provide palatable oral preparations. Such suspension may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Pharmaceutical compositions may also be in the form of oil-in-water emulsions. The oily phase may be a vegetable oil (e.g., olive oil or arachis oil) or a mineral oil (e.g., liquid paraffin) or mixtures thereof. Suitable emulsifying agents may be naturally-occurring gums (e.g., gum acacia or gum tragacanth), naturally-occurring phosphatides (e.g., soy bean, lecithin, and esters or partial esters derived from fatty acids and hexitol), anhydrides (e.g., sorbitan monoleate) and condensation products of partial esters derived from fatty acids and hexitol with ethylene oxide (e.g., polyoxyethylene sorbitan monoleate). The emulsions may also contain sweetening and/or flavoring agents.

Syrups and elixirs may be formulated with sweetening agents, such as glycerol, propylene glycol, sorbitol or sucrose. Such formulations may also comprise one or more demulcents, preservatives, flavoring agents and/or coloring agents.

A pharmaceutical composition may be prepared as a sterile injectible aqueous or oleaginous suspension. The MCH receptor antagonist, depending on the vehicle and concentration used, can either be suspended or dissolved in the vehicle. Such a composition may be formulated according to the known art using suitable dispersing, wetting agents and/or suspending agents such as those mentioned above. Among the acceptable vehicles and solvents that may be employed are water, 1,3-butanediol, Ringer's solution and isotonic sodium chloride solution. In addition, sterile, fixed oils may be employed as a solvent or suspending medium. For this purpose any bland fixed oil may be employed, including synthetic mono- or diglycerides. In addition, fatty acids such as oleic acid find use in the preparation of injectible compositions, and adjuvants such as local anesthetics, preservatives and/or buffering agents can be dissolved in the vehicle.

Compositions may also be prepared in the form of suppositories (e.g., for rectal administration). Such compositions can be prepared by mixing the drug with a suitable non-irritating excipient that is solid at ordinary temperatures but liquid at the rectal temperature and will therefore melt in the rectum to release the drug. Suitable excipients include, for example, cocoa butter and polyethylene glycols.

For administration to non-human animals, the composition may also be added to animal feed or drinking water. It may be convenient to formulate animal feed and drinking water compositions so that the animal takes in an appropriate quantity of the composition along with its diet. It may also be convenient to present the composition as a premix for addition to feed or drinking water.

Pharmaceutical compositions may be formulated as sustained release formulations (i.e., a formulation such as a capsule that effects a slow release of MCH receptor antagonist following administration). Such formulations may generally be prepared using well known technology and administered by, for example, oral, rectal or subcutaneous implantation, or by implantation at the desired target site. Carriers for use within such formulations are biocompatible, and may also be biodegradable; preferably the formulation provides a relatively constant level of MCH receptor antagonist release. The amount of antagonist contained within a sustained release formulation depends upon the site of implantation, the rate and expected duration of release and the nature of the condition to be treated or prevented.

MCH receptor antagonists are generally present within a pharmaceutical composition in a therapeutically effective amount. A therapeutically effective amount is an amount that results in a discernible benefit in a patient carrying an MC4R mutation. Such benefit(s) include one or more of decreased BMI, decreased food intake and/or weight loss, following repeated administration (e.g., from 1 to 4 times per day for a period of weeks or months). A preferred concentration is one sufficient to inhibit the binding of MCH to MCHR1 receptor in vitro. Compositions providing dosage levels ranging from about 0.1 mg to about 140 mg per kilogram of body weight per day are preferred (about 0.5 mg to about 7 g per human patient per day). The amount of active ingredient that may be combined with the carrier materials to produce a single dosage form will vary depending upon the host treated and the particular mode of administration. Dosage unit forms will generally contain from about 1 mg to about 500 mg of an active ingredient. It will be understood, however, that the optimal dose for any particular patient will depend upon a variety of factors, including the activity of the specific compound employed; the age, body weight, general health, sex and diet of the patient; the time and route of administration; the rate of excretion; any simultaneous treatment, such as a drug combination; and the type and severity of the particular disease undergoing treatment. Optimal dosages may be established using routine testing, and procedures that are well known in the art.

Methods of Use

The present invention provides methods for preventing treating health conditions associated with MC4R mutations, such as obesity and overeating, and for reducing the body mass index of a patient carrying at least one MC4R mutation. Obesity and overeating may be diagnosed and monitored using criteria that have been established in the art. Patients may include humans, domesticated companion animals (pets, such as dogs) and livestock animals, and are typically obese at the time of initiating treatment.

In general, prior to treatment, a determination is made as to whether or not the patient carries at least one MC4R mutation, as defined above. Such mutations include, but are not limited to, deletion of CTCT at codon 211, insertion of four nucleotides at codon 244, nonsense mutation at position 35, and missense mutations (e.g., Thr11Ser, Arg18Cys, Ser30Phe, Asp37Val, Val50Met, SerS8Cys, Pro78Leu, Gly98Arg, Ile102Ser, Val103Ile, Thr112Met, Ile137Thr, Thr150Ile, Arg165Trp, Ile170Val, Leu250Gln, Gly252Ser, Asn274Ser, Ile301Thr and/or Ue317Thr).

A determination as to whether or not the patient carries at least one MC4R mutation may be made by review of the patient's chart, or using standard diagnostic methods. As an initial screen, a patient may (but need not) be evaluated for characteristics commonly associated with MC4R mutations, including early onset obesity associated with hyperphagia, tall stature, high blood pressure, hyperinsulinemia in the absence of diabetes and preserved reproductive function. The presence of an MC4R mutation may be determined, for example, via PCR assay, in which an MC4R nucleotide sequence in a sample (e.g., tissue or body fluid) obtained from a patient is compared to a reference MC4R sequence for the patient's species. Suitable PCR assays will be apparent to those of ordinary skill in the art and include, for example, assays described by Hinney et al. (1999) J. Clin. Endocrinology and Metabolism 84:1483-86 and Vaisse et al. (2000) J. Clin. Invest 106:253-62. It will be apparent that this determination does not require a comparison of the complete MC4R sequences. Rather, the determination may be made by simply assaying the patient's MC4R nucleotide sequence(s) for the presence of a specific nucleotide or series of nucleotides that are associated with obesity.

Patients who carry at least one MC4R mutations may be obese or nonobese (i.e., have never been obese or were previously obese). In either case, therapy involves administering a non-toxic melanin concentrating hormone (MCH) receptor antagonist to the patient, with dosages generally as described above. For an obese patient, the amount administered is generally an amount that is effective to reduce (1) food consumption and/or (2) body mass index of the patient upon sustained administration. In other words, the amount in one dose need not have a detectable effect on body mass index; however, when administered repeatedly as described herein, the amount should be sufficient to detectably reduce food consumption and/or body mass index. For patients who are not currently obese, methods provided herein generally prevent obesity (i.e., therapy results in a decrease in the amount of weight gained, a delay in the onset of weight gain or a maintenance of the patient's current weight). An effective amount is generally an amount that is found in clinical trials to decrease overeating and/or to prevent, decrease or delay the onset of weight gain in patients that carry one or more MC4R mutations.

Frequency of dosage may vary depending on the compound used and the particular condition to be treated. In general, a dosage regimen of 4 times daily or less is preferred, with 1 or 2 times daily particularly preferred. The specific dose for any particular patient will depend upon a variety of factors discussed above. In general, the use of the minimum dosage that is sufficient to provide effective therapy is preferred. Patients may generally be monitored for therapeutic effectiveness using assays suitable for the condition being treated or prevented, which will be familiar to those of ordinary skill in the art. For example, treatment is considered to be effective if it results in a statistically significant decrease in weight, BMI or food intake.

The following Examples are offered by way of illustration and not by way of limitation. Unless otherwise specified all reagents and solvents are of standard commercial grade and are used without further purification.

### EXAMPLES

### Example 1

### Effect of MCH Receptor Antagonist on Food Consumption Stimulated by Reduced MC4 Receptor Activity

This Example illustrates an in vivo assay for use in confirming the ability of a MCH receptor antagonist to inhibit excess food consumption resulting from decreased MC4 activity.

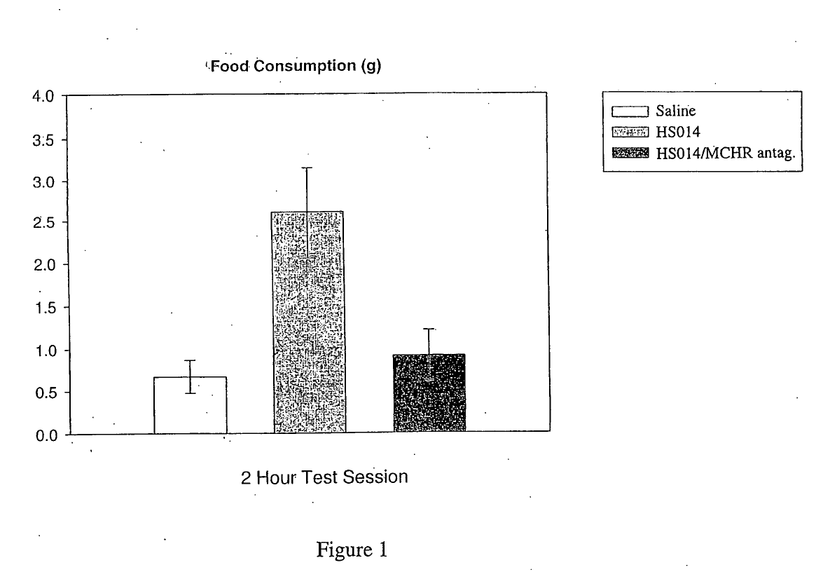

Experimentally naïve male Sprague Dawley rats (Sasco, St. Louis, Mo.) weighing between 250 and 300 grams are housed in stainless steel hanging cages in a temperature and humidity controlled animal facility (22±2° C., 40-70% relative humidity) with a 12 hour light/dark cycle. Rats are implanted with a 26 g stainless steel cannula aimed at the lateral ventricle. After one week of recovery, 5, 10 or 20 mg/kg MCH receptor antagonist is administered orally in 2% d-α-tocopherol polyethylene glycol succinate to test animals (with vehicle alone administered to control animals) 30 minutes before ICV administration of 6 nmol HS014 (Phoenix Peptide (Belmont, Calif.); dissolved in distilled water) or distilled water vehicle in a volume of 5 μL. Rats are then placed in their home cages and allowed free access to pre-weighed Purina chow pellets and water. Food consumption is measured 2 hours post ICV injection, and the results are shown in FIG. 1.

A one-way ANOVA is conducted on the food consumption measurements. Significant dose effects (p<0.05) are further analyzed using a Fisher LSD test. Animals that receive HS014 (a cyclic analogue of MSH that functions as a selective MC4 receptor antagonist) eat significantly more food than animals that receive an ICV injection of water vehicle (p<0.05). Animals administered HS014 and MCH receptor antagonist eat significantly less than animals that receive HS014 alone (p<0.05). Preferably, the level of food consumption in animals treated with HS014 and 20 mg/kg MCH receptor antagonist is not significantly different from the level of consumption in animals treated with vehicle alone (i.e., without HS014).

### Example 2

### Melanin Concentrating Hormone Receptor Binding Assay

This Example illustrates a standard assay of melanin concentrating hormone receptor binding that may be used to determine the binding affinity of compounds for the MCH receptor.

MCH1-containing membranes are prepared as described at pages 48-49 of WO 03/060475. Competition binding assays are performed at room temperature in Falcon 96 well round bottom polypropylene plates. Each assay well contains 150 μl of MCH1-containing membranes prepared as described above, 50 μl ¹²⁵I-Tyr MCH, 50 μl binding buffer, and 2 μl test compound in DMSO. ¹²⁵I-Tyr MCH (specific activity=2200 Ci/mMol) is purchased from NEN, Boston, Mass. (Cat # NEX 373) and is diluted in binding buffer to provide a final assay concentration of 30 pM.

Non-specific binding is defined as the binding measured in the presence of 1 μM unlabeled MCH. MCH is purchased from BACHEM U.S.A., King of Prussia, Pa. (cat # H-1482). Assay wells used to determine MCH binding contained 150 μl of MCH receptor containing membranes, 50 μl ¹²⁵I-Tyr MCH, 25 μl binding buffer, and 25 μl binding buffer.

Assay plates are incubated for 1 hour at room temperature. Membranes are harvested onto WALLAC™ glass fiber filters (PERKIN-ELMER, Gaithersburg, Md.) which are pre-soaked with 1.0% PEI (polyethyleneimine) for 2 hours prior to use. Filters are allowed to dry overnight, and then counted in a WALLAC 1205 BETA PLATE counter after addition of WALLAC BETA SCINT™ scintillation fluid.

For saturation binding, the concentration of ¹²⁵I-Tyr MCH is varied from 7 to 1,000 pM. Typically, 11 concentration points are collected per saturation binding curve. Equilibrium binding parameters are determined by fitting the allosteric Hill equation to the measured values with the aid of the computer program FitP™ (BIOSOFT, Ferguson, Mo.). For the compounds described herein, Kᵢ values are below 1 micromolar, preferably below 500 nanomolar, more preferably below 100 nanomolar.

### Example 3

### Calcium Mobilization Assay

This Example illustrates a representative functional assay for monitoring the response of cells expressing melanin concentrating hormone receptors to melanin concentrating hormone. This assay can also be used to determine if test compounds act as agonists or antagonists of melanin concentrating hormone receptors.

Chinese Hamster Ovary (CHO) cells (American Type Culture Collection; Manassas, Va.) are stably transfected with an MCH receptor expression vector as described at page 50 of WO 03/060475, and are grown to a density of 15,000 cells/well in FALCON™ black-walled, clear-bottomed 96-well plates (#3904, BECTON-DICKINSON, Franklin Lakes, N.J.) in Ham's F12 culture medium (MEDIATECH, Herndon, Va.) supplemented with 10% fetal bovine serum, 25 mM HEPES and 500 μg/mL (active) G418. Prior to running the assay, the culture medium is emptied from the 96 well plates. Fluo-3 calcium sensitive dye (Molecular Probes, Eugene, Oreg.) is added to each well (dye solution: 1 mg FLUO-3 AM, 440 μL DMSO and 440 μl 20% pluronic acid in DMSO, diluted 1:4, 50 μl diluted solution per well). Plates are covered with aluminum foil and incubated at 37° C. for 1-2 hours. After the incubation, the dye is emptied from the plates, cells are washed once in 100 μl KRH buffer (0.05 mM KCl, 0.115 M NaCl, 9.6 mM NaH₂PO₄, 0.01 mM MgSO₄, 25 mM HEPES, pH 7.4) to remove excess dye; after washing, 80 μl KRH buffer is added to each well. Fluorescence response is monitored upon the addition of either human MCH receptor or test compound by a FLIPR™ plate reader (Molecular Devices, Sunnyvale, Calif.) by excitation at 480 nM and emission at 530 nM.

In order to measure the ability of a test compound to antagonize the response of cells expressing MCH receptors to MCH, the EC₅₀ of MCH is first determined. An additional 20 μl of KRH buffer and 1 μl DMSO is added to each well of cells, prepared as described above. 100 μl human MCH in KRH buffer is automatically transferred by the FLIPR instrument to each well. An 8-point concentration response curve, with final MCH concentrations of 1 nM to 3 μM, is used to determine MCH EC₅₀.

Test compounds are dissolved in DMSO, diluted in 20 μl KRH buffer, and added to cells prepared as described above. The 96 well plates containing prepared cells and test compounds are incubated in the dark, at room temperature for 0.5-6 hours. It is important that the incubation not continue beyond 6 hours. Just prior to determining the fluorescence response, 100 μl human MCH diluted in KRH buffer to 2×EC₅₀ is automatically added by the FLIPR instrument to each well of the 96 well plate for a final sample volume of 200 μl and a final MCH concentration of EC₅₀. The final concentration of test compounds in the assay wells is between 1 μM and 5 μM. Typically, cells exposed to one EC₅₀ of MCH exhibit a fluorescence response of about 10,000 Relative Fluorescence Units. Antagonists of the MCH receptor exhibit a response that is significantly less than that of the control cells to the p≦0.05 level, as measured using a parametric test of statistical significance. Typically, antagonists of the MCH receptor decrease the fluorescence response by about 20%, preferably by about 50%, and most preferably by at least 80% as compared to matched controls.

The ability of a compound to act as an agonist of the MCH receptor is determined by measuring the fluorescence response of cells expressing MCH receptors, using the methods described above, in the absence of MCH. Compounds that cause cells to exhibit fluorescence above background are MCH receptor agonists. Compounds that induce no detectable increase in the basal activity of the MCH receptor have no detectable agonist activity and are preferred.

### Example 4

### MDCK Cytotoxicity Assay

This Example illustrates the evaluation of compound toxicity using a Madin Darby canine kidney (MDCK) cell cytoxicity assay.

1 μL of test compound is added to each well of a clear bottom 96-well plate (PACKARD, Meriden, Conn.) to give final concentration of compound in the assay of 10 micromolar, 100 micromolar or 200 micromolar. Solvent without test compound is added to control wells.

MDCK cells, ATCC no. CCL-34 (American Type Culture Collection, Manassas, Va.), are maintained in sterile conditions following the instructions in the ATCC production information sheet. Confluent MDCK cells are trypsinized, harvested, and diluted to a concentration of 0.1×10⁶ cells/ml with warm (37° C.) medium (VITACELL Minimum Essential Medium Eagle, ATCC catalog # 30-2003). 100 μL of diluted cells is added to each well, except for five standard curve control wells that contain 100 μL of warm medium without cells. The plate is then incubated at 37° C. under 95% O₂, 5% CO₂ for 2 hours with constant shaking. After incubation, 50 μL of mammalian cell lysis solution is added per well, the wells are covered with PACKARD TOPSEAL stickers, and plates are shaken at approximately 700 rpm on a suitable shaker for 2 minutes.

Compounds causing toxicity will decrease ATP production, relative to untreated cells. The PACKARD, (Meriden, Conn.) ATP-LITE-M Luminescent ATP detection kit, product no. 6016941, is generally used according to the manufacturer's instructions to measure ATP production in treated and untreated MDCK cells. PACKARD ATP LITE-M reagents are allowed to equilibrate to room temperature. Once equilibrated, the lyophilized substrate solution is reconstituted in 5.5 mL of substrate buffer solution (from kit). Lyophilized ATP standard solution is reconstituted in deionized water to give a 10 mM stock. For the five control wells, 10 μL of serially diluted PACKARD standard is added to each of the standard curve control wells to yield a final concentration in each subsequent well of 200 nM, 100 nM, 50 nM, 25 nM and 12.5 nM. PACKARD substrate solution (50 μL) is added to all wells, which are then covered, and the plates are shaken at approximately 700 rpm on a suitable shaker for 2 minutes. A white PACKARD sticker is attached to the bottom of each plate and samples are dark adapted by wrapping plates in foil and placing in the dark for 10 minutes. Luminescence is then measured at 22° C. using a luminescence counter (e.g., PACKARD TOPCOUNT Microplate Scintillation and Luminescence Counter or TECAN SPECTRAFLUOR PLUS), and ATP levels calculated from the standard curve. ATP levels in cells treated with test compound(s) are compared to the levels determined for untreated cells. Cells treated with 10 μM of a preferred test compound exhibit ATP levels that are at least 80%, preferably at least 90%, of the untreated cells. When a 100 μM concentration of the test compound is used, cells treated with preferred test compounds exhibit ATP levels that are at least 50%, preferably at least 80%, of the ATP levels detected in untreated cells.

From the foregoing it will be appreciated that, although specific embodiments of the invention have been described herein for purposes of illustration, various modifications may be made without deviating from the spirit and scope of the invention.

1. A method for treating obesity in a mammalian patient, comprising determining whether or not the patient carries at least one melanocortin 4 (MC4) receptor mutation that is associated with obesity and, if the patient carries such a mutation, administering an amount of a non-toxic melanin concentrating hormone (MCH) receptor antagonist effective to reduce either or both of (1) food consumption or (2) body mass index of the patient upon sustained administration.

2. A method according to claim 1, wherein the MCH receptor antagonist has a molecular mass less than 700 a.m.u. and is nonpeptidic.

3. A method according to claim 1, wherein the MCH receptor antagonist has no detectable MCH receptor agonist activity.

4. A method according to claim 1, wherein the MCH receptor antagonist binds to an MCH receptor with a Kᵢ that is less than 1 micromolar.

5. A method according to claim 1, wherein the MCH receptor antagonist binds to an MCH receptor with a Kᵢ that is less than 100 nanomolar.

6. A method according to claim 1, wherein the MCH receptor antagonist is administered orally.

7. A method according to claim 1, wherein the MCH receptor antagonist is administered by injection.

8. A method according to claim 1, wherein the determination of whether or not the patient carries an MC4 receptor mutation is performed via PCR using a sample of a tissue or body fluid obtained from the patient.

9. A method for treating obesity in a patient carrying at least one MC4 receptor mutation that is associated with obesity, comprising administering an effective amount of a non-toxic MCH receptor antagonist to a patient previously determined to carry such a mutation.

10. A method according to claim 9, wherein the MCH receptor antagonist has a molecular mass less than 700 a.m.u. and is nonpeptidic.

11. A method according to claim 9, wherein the MCH receptor antagonist has no detectable MCH receptor agonist activity.

12. A method according to claim 9, wherein the MCH receptor antagonist binds to an MCH receptor with a Kᵢ that is less than 1 micromolar.

13. A method according to claim 9, wherein the MCH receptor antagonist binds to an MCH receptor with a Kᵢ that is less than 100 nanomolar.

14. A method according to claim 9, wherein the MCH receptor antagonist is administered orally.

15. A method according to claim 9, wherein the MCH receptor antagonist is administered by injection.

16. A method for preventing obesity in a mammalian patient, comprising determining whether or not the patient carries at least one MC4 receptor mutation that is associated with obesity and, if the patient carries such a mutation, administering an effective amount of a non-toxic melanin concentrating hormone (MCH) receptor antagonist, and thereby preventing obesity in the patient.

17. A method according to claim 16, wherein the MCH receptor antagonist has a molecular mass less than 700 a.m.u. and is nonpeptidic.

18. A method according to claim 16, wherein the MCH receptor antagonist has no detectable MCH receptor agonist activity.

19. A method according to claim 16, wherein the MCH receptor antagonist binds to an MCH receptor with a Kᵢ that is less than 1 micromolar.

20. A method according to claim 16, wherein the MCH receptor antagonist binds to an MCH receptor with a Kᵢ that is less than 100 nanomolar.

21. A method according to claim 16, wherein the MCH receptor antagonist is administered orally.

22. A method according to claim 16, wherein the MCH receptor antagonist is administered by injection.

23. A method according to claim 16, wherein the determination of whether or not the patient carries an MC4 receptor mutation is performed via PCR using a sample of a tissue or body fluid obtained from the patient.

24. (canceled)


SEQUENCE LISTING (1 sequence)

SEQ ID NO: 1 - protein, 332 aa, Homo Sapiens
MVNSTHRGMHTSLHLWNRSSYRLHSNASESLGKGYSDGGCYEQLFVSPEVFVTLGVISLLENILVIVAIAKNKNLHSPMYFFICSLAVADMLVSVSNGSETIVITLLNSTDTDAQSFTVNIDNVIDSVICSSLLASICSLLSIAVDRYFTIFYALQYHNIMTVKRVGISISCIWAACTVSGILFIIYSDSSAVIICLITMFFTMLALMASLYVHMFLMARLHIKRIAVLPGTGAIRQGANMKGAITLTILIGVFVVCWAPFFLHLIFYISCPQNPYCVCFMSHFNLYLILIMCNSIIDPLIYALRSQELRKTFKEIICCYPLGGLCDLSSRY